(19) United States
(12) Patent Application Publication
(10) Pub. No.: US 2011/0155164 A1
(43) Pub. Date: Jun. 30, 2011

(54) COSMETIC COMPOSITION COMPRISING AT LEAST ONE ORGANIC SILICON COMPOUND, AT LEAST ONE ANIONIC SURFACTANT AND AT LEAST ONE AMINATED SILICONE AS WELL AS A METHOD EMPLOYING SAID COMPOSITION

(76) Inventors: Valérie VIRAVAU, Paris (FR); Carine Aires, Paris (FR); Damien Drillon, Paris (FR); Myriam Mellul, Paris (FR)

(21) Appl. No.: 12/977,478

(22) Filed: Dec. 23, 2010

Related U.S. Application Data
(60) Provisional application No. 61/298,294, filed on Jan. 26, 2010.

(30) Foreign Application Priority Data
Dec. 23, 2009 (FR) 0959488

(51) Int. Cl. A61Q 5/12; A61Q 5/02; A61K 8/58; A61K 8/72

(52) U.S. Cl. 132/202; 424/70.22; 424/70.24; 424/70.122; 424/70.19

(57) ABSTRACT

The present disclosure relates to cosmetic compositions for washing and conditioning keratin fibers, such as human keratin fibers, for example, the hair, comprising in a cosmetically acceptable medium: i) at least one organic silicon compound chosen from silanes comprising one silicon atom and siloxanes comprising two or three silicon atoms, said organic silicon compounds having at least one basic chemical functional group and at least one group chosen from hydroxyl groups and hydrolysable groups per molecule; ii) at least one anionic surfactant; and iii) at least one aminated silicone not having a quaternary ammonium group different from the at least one organic silicon compound of part i). The present disclosure also relates to a method of cosmetic treatment of keratin fibers as well as a use employing a cosmetic composition according to the disclosure.

This application claims benefit of U.S. Provisional Application No. 61/298,294, filed Jan. 26, 2010. This application also claims benefit of priority under 35 U.S.C. §119 to French Patent Application No. 0959488, filed Dec. 23, 2009.

The present disclosure relates to a composition for cleaning and conditioning keratin fibers, including human keratin fibers such as the hair, comprising, in a cosmetically acceptable medium, at least one organic silicon compound, at least one anionic surfactant and at least one aminated silicone different from the at least one organic silicon compound. The present disclosure also relates to a method of cosmetic treatment of keratin fibers.

Detergent compositions (such as shampoos) essentially based on conventional surfactants notably of the anionic, non-ionic and/or amphoteric type, for instance of the anionic type, are commonly used for cleaning and/or washing keratinous materials such as the hair. These compositions are applied on the wetted hair and the foam generated by massaging or rubbing with the hands can make it possible, after rinsing with water, to remove the various kinds of dirt initially present on the hair or on the skin.

These basic compositions can provide good washing power but their intrinsic cosmetic properties can still be fairly poor; the character of this kind of cleaning treatment may be relatively aggressive and can eventually lead to damage of the hair fiber to a varying degree, which may be associated with the progressive removal of the lipids or proteins contained in or on the surface of the latter.

Moreover, to improve the cosmetic properties of the aforementioned detergent compositions, such as those that are intended to be applied on sensitized hair (i.e. hair that has generally been damaged or weakened by the action of outdoor atmospheric agents such as light and bad weather, and/or of mechanical or chemical treatments such as brushing, combing, dyeing, bleaching, perming and/or straightening), supplementary cosmetic agents called conditioners may be incorporated, which are intended mainly to repair or limit the harmful or undesirable effects caused by the various treatments or aggressive factors to which the hair fibers are subjected, more or less repeatedly. These conditioners can of course also improve the cosmetic properties of natural hair.

With this aim, the use of cosmetically active organic compounds has already been proposed, such as cationic polymers and silicones as conditioners, in detergent cosmetic compositions such as shampoos, to endow the hair with satisfactory cosmetic properties, such as of sheen, softness, suppleness, lightness, a natural feel as well as an improved capacity for disentangling.

However, the use of these compounds in cosmetic compositions for washing and conditioning may not provide the hair with satisfactory and sufficiently lasting styling properties. In fact, the styling effects that may be provided by these compositions, such as effects of form retention, body and/or of manageability of the hair, can be inadequate, for example, tending to fade after washing the hair with a conventional shampoo.

There seems to be increasing demand among consumers for washing compositions that are not only capable of providing suitable conditioning of the hair, but are also capable of providing satisfactory and lasting styling effects.

Thus, compositions intended for hair washing and conditioning, which may comprise organic silicon compounds, such as 3-aminopropyltriethoxysilane, have been developed in response to these demands. These washing compositions can provide conditioning of the hair, and may give it a soft feel, while conferring durable styling effects.

Moreover, these compositions can facilitate the shaping of fine hair and impart styling effects to curly hair with respect to the shape and control of the curls.

However, washing compositions comprising these organic silicon compounds may have the drawback that they change considerably over time in normal conditions of storage depending on the temperature, e.g., with respect to their viscosity and their appearance. In other words, these compositions can have low stability, which can be reflected in a cloudy appearance as well as an unsatisfactory texture on storage.

In fact, it was found that organic silicon compounds, such as 3-aminopropyltriethoxysilane, can exhibit chemical incompatibility with some surfactants, including at least some anionic surfactants, that may be present in washing compositions, which can cause the stability problems that are encountered.

Moreover, it has been observed that the inclusion of certain organic silicon compounds, such as aminated derivatives, e.g., 3-aminopropyltriethoxysilane, in washing compositions, which generally have a pH ranging from 4 to 7, can also give rise to stability problems owing to the alkaline character of these compounds.

There is therefore a real need to develop cosmetic compositions intended for cleaning and conditioning keratin fibers that contain organic silicon compounds, with said compositions avoiding or reducing at least some of the drawbacks described above, i.e. which can provide beneficial properties such as being stable over time and can provide satisfactory hair conditioning while also providing powerful and durable styling effects, e.g., in terms of at least one of weight, body, and texturing of the hair.

This disclosure is based in part on the finding that it was possible to formulate detergent and conditioning compositions having beneficial properties, comprising at least one organic silicon compound as defined below, at least one anionic surfactant and at least one non-quaternized aminated silicone different from the at least one organic silicon compound.

The use of aminated silicones that do not have a quaternary ammonium group in their structure in cosmetic compositions comprising at least one organic silicon compound and at least one anionic surfactant can make the compositions stable in storage both at room temperature (20-25° C.) and at 45° C., e.g., with respect to their appearance and/or their viscosity.

“Stable” means, in the sense of the present disclosure, that both the appearance and the viscosity of these compositions do not change appreciably over time in the standard conditions of storage tests, for example for 2 months at room temperature (20° C.-25° C.), and/or at 45° C. and/or at 4° C., following their manufacture.

Moreover, the compositions according to the disclosure can lead to satisfactory treatment of the hair, imparting, e.g., a satisfactory soft feel, improved capacity for disentangling, softness and/or suppleness.

Moreover, the compositions according to the present disclosure can provide the hair with powerful styling effects, e.g., giving it weight, body and/or manageability, and the effects can be long-lasting.

Moreover, the compositions according to the disclosure can facilitate shaping of the hair, such as fine hair, and/or impart improved styling effects to curly hair, e.g., in terms of the shape and control of the curls, and the effects can be long-lasting.

The present disclosure relates in part to a cosmetic composition for washing and conditioning keratin fibers, e.g., human keratin fibers such as the hair, comprising in a cosmetically acceptable medium:

i) at least one organic silicon compound chosen from silanes comprising one silicon atom and siloxanes comprising two or three silicon atoms, said at least one organic silicon compound having at least one basic chemical functional group and at least one group chosen from hydroxyl groups and hydrolysable groups per molecule;

ii) at least one anionic surfactant; and

iii) at least one non-quaternized aminated silicone different from the at least one organic silicon compound of part i).

The present disclosure also relates to a method of cosmetic treatment of the hair, such as washing and conditioning, comprising application of a composition according to the disclosure on said fibers.

It also relates to the use of the compositions according to the disclosure as shampoo for hair washing and conditioning.

Other objects and characteristics, aspects and benefits of the disclosure will become clearer on reading the description and examples given below.

The at least one organic silicon compound used in compositions according to the disclosure is chosen from organosilanes comprising one silicon atom and organosiloxanes having two or three silicon atoms. They further comprise at least one basic chemical functional group, and may comprise only a single basic chemical functional group. The basic chemical functional group can correspond to any functional group imparting a basic character to the silicon compound, for example, an amine functional group such as a primary, secondary or tertiary amine functional group. The silicon compounds according to the disclosure can optionally comprise other functional groups, for example an acid functional group or a halogen functional group.

The organic silicon compound or compounds used in the compositions according to the disclosure further comprises at least one or at least two hydrolysable groups or hydroxyl groups per molecule. The hydrolysable groups can be, for example, alkoxy, aryloxy or halogen groups. They can also optionally comprise other chemical functional groups such as acid functional groups.

In some embodiments, the organosilane or organosilanes used in the compositions according to the disclosure are chosen from compounds of formula (I):

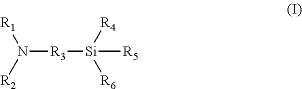

in which:

R₄ represents a halogen, OR′ or R′₁;

R₅ represents a halogen, OR″ or R′₂;

R₆ represents a halogen, OR″ or R′₃;

R₁, R₂, R₃, R′, R″, R′″, R′₁, R′₂, R′₃ represent, independently of one another, a saturated or unsaturated, linear or branched hydrocarbon group, optionally bearing additional chemical groups, and R₁, R₂, R′, R″ and R′″ can moreover denote hydrogen, and at least two of the groups R₄, R₅ and R₆ denote OR′, OR″ and OR′″ respectively, at least two of the groups R′, R″ and R′″ being different from hydrogen.

In some embodiments, the groups R₁, R₂, R′, R₁, R′₂, R′₃, R″ and R′″ are chosen from C₁-C₁₂ alkyl, C₆ to C₁₄ aryl, C₁ to C₈ alkyl-C₆ to C₁₄ aryl, and C₆ to C₁₄ aryl-C₁ to C₈ alkyl radicals.

In some embodiments, the at least one organosiloxane used in the compositions according to the disclosure is chosen from compounds of formula (II):

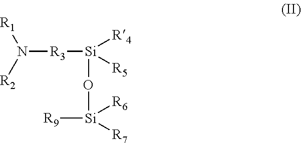

in which:

R₁, R₂, R₃, R₅ and R₆ are defined as previously;

R′₄ represents a halogen atom or ORᵢᵢ;

R₇ represents a halogen atom, OR₁₀ or R″₁;

R₉ represents a halogen atom, OR₈, R″₂ or R₃NR₁R₂;

R″₁, R″₂, R₈, R₁₀ and R₁₁ represent a saturated or unsaturated, linear or branched hydrocarbon group, optionally bearing additional chemical groups, and the groups R₁₁, R₁₀ and R₈ can moreover represent a hydrogen atom; at least one of groups R₆, R₇ and R₉ denoting a halogen atom, OR″″, OR₁₀ or OR₈.

In some embodiments, groups R″₁, R″₂, R₆ or R₁₀ and R₁₁ are chosen from C₁-C₁₂ alkyl, C₆ to C₁₄ aryl, C₁ to C₈ alkyl-C₆ to C₁₄ aryl, and C₆ to C₁₄ aryl-Cᵢ to C₈ alkyl radicals.

The halogen atom can be, for example, a chlorine atom.

The organic silicon compound or compounds used in the compositions according to the disclosure can be an organosilane or organosilanes chosen from compounds of formula (III):

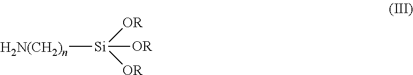

in which the radicals R, which may be identical or different, are chosen from C₁-C₆ or C₁-C₂ alkyl radicals, and n is an integer from 1 to 6, such as from 2 to 4.

In some embodiments, the at least one organic silicon compound is soluble in water, for example, at a concentration of at least 2 wt. %, a concentration of at least 5 wt. %, or a concentration of at least 10 wt. % in water at a temperature of 25° C.±5° C., for example, 20° C., 25° C., or 30° C., and at atmospheric pressure, for example, 1 atm. “Soluble” means that a single macroscopic phase is formed.

In some embodiments, the at least one organic silicon compound present in the compositions according to the disclosure comprises or consists of 3-aminopropyltriethoxysilane.

The at least one organic silicon compound can be present in the compositions according to the disclosure at a content ranging from 0.01 to 10 wt. %, such as at a content by weight ranging from 0.1 to 5 wt. %, or at a content by weight ranging from 0.2 to 2 wt. %, relative to the total weight of the composition.

As stated previously, the cosmetic compositions according to the present disclosure further contain at least one anionic surfactant.

The at least one anionic surfactant used in the compositions of the disclosure can be chosen from salts, including without limitation salts of alkali metals such as sodium salts, ammonium salts, salts of amines, salts of amino alcohols or salts of alkaline-earth metals, for example, of magnesium, of the following types: alkylsulphates, alkylethersulphates, alkylamidoethersulphates, alkylarylpolyethersulphates, monoglyceridesulphates, alkylsulphonates, alkylamidesulphonates, alkylarylsulphonates, α-olefinsulphonates, paraffin-sulphonates, alkylsulphosuccinates, alkylethersulphosuccinates, alkylamide-sulphosuccinates, alkylsulpho-acetates, acylsarcosinates and acylglutamates, the alkyl and acyl groups of all of these compounds having from 6 to 24 carbon atoms; in some embodiments, the aryl group is a phenyl or benzyl group.

It is also possible to use the monoesters of C₆₋₂₄ alkyl and of polyglycoside-dicarboxylic acids such as alkyl glucoside-citrates, alkyl polyglycoside-tartrates and alkyl polyglycoside-sulphosuccinates, alkylsulphosuccinamates, acylisethionates and N-acyltaurates, the alkyl or acyl group of all of these compounds having from 12 to 20 carbon atoms.

The at least one anionic surfactant usable in the compositions of the present disclosure also include the acyl-lactylates whose acyl group has from 8 to 20 carbon atoms.

The at least one anionic surfactant usable in the compositions of the present disclosure also include the alkyl-D-galactoside-uronic acids and salts thereof as well as (C₆₋₂₄ alkyl)ether-carboxylic polyalkoxylated acids, (C₆₋₂₄ alkyl)(C₆₋₂₄ aryl)ether-carboxylic polyalkoxylated acids, (C₆₋₂₄ alkyl)amidoether-carboxylic polyalkoxylated acids and salts thereof, such as those having from 2 to 50, from 2 to 10, or from 2 to 5 ethylene oxide units, and mixtures thereof.

It is possible to use alkylsulphates, alkylethersulphates, alkylethercarboxylates and mixtures thereof, such as in the form of salts of alkali metals or alkaline-earth metals, of ammonium, of amines or of amino alcohols.

In some embodiments, the at least one anionic surfactant used in the cosmetic compositions according to the disclosure are chosen from alkylethersulphates, for example C₁₂-C₁₄ alkylethersulphates (it is also possible for the alkylethersulphates to comprise 2 to 3 moles of ethylene oxide), and the N-acyl taurates.

The at least one anionic surfactant can be present at a content ranging from 1 to 25 wt. %, e.g., at a content ranging from 3 to 20 wt. %, or at a content ranging from 5 to 15 wt. %, relative to the total weight of the cosmetic composition according to the disclosure.

The compositions according to the present disclosure further contain at least one non-quaternized aminated silicone different from the at least one silane or siloxane (i) also present in the compositions.

“Non-quaternized aminated silicone” means, in the sense of the present disclosure, an aminated silicone that does not have quaternized ammonium groups.

In other words, the aminated silicone or silicones used in the cosmetic compositions according to the present disclosure have in their structure at least one primary, secondary or tertiary amine functional group but do not have a quaternary ammonium functional group.

In some embodiments, the aminated silicone or silicones used in the cosmetic compositions according to the present disclosure have in their structure at least 4 silicon atoms.

Herein, “silicone” denotes, in accordance with general usage, all organosilicon polymers or oligomers with a linear or cyclic, branched or crosslinked structure, of variable molecular weight, obtained by polymerization and/or by polycondensation of suitably functionalized silanes, and constituted essentially of a repetition of main units in which the silicon atoms are joined together by oxygen atoms (siloxane bond —Si—O—Si—), hydrocarbon radicals optionally substituted, being directly bound by a carbon atom to said silicon atoms. Common hydrocarbon radicals include alkyl radicals, e.g., of C₁-C₁₀, such as methyl; fluoroalkyl radicals whose alkyl portion is C₁-C₁₀; and aryl radicals, e.g., phenyl.

In some embodiments, the at least one aminated silicone used in the compositions according to the present disclosure is chosen from:

(a) compounds of formula (IV):

(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (IV)

in which,

T is a hydrogen atom, or a phenyl, hydroxyl (—OH), or C₁-C₈ alkyl radical, e.g., methyl or C₁-C₈ alkoxy, such as methoxy,

a denotes an integer from zero to 3,

b denotes zero or 1,

m and n are numbers that sum to a value ranging from 1 to 2000, e.g., from 50 to 150; in some embodiments, n can denote a number from 0 to 1999, or from 49 to 149, and/or m can denote a number from 1 to 2000, or from 1 to 10;

R₁ is a monovalent radical of formula —C_{q}H_{2q}L in which q is a number from 2 to 8 and L is an amino group chosen from the groups:

—N(R²)—CH₂—CH₂—N(R²)₂;

—N(R²)₂;

in which R² can denote a hydrogen atom, a phenyl radical, a benzyl radical, or a saturated monovalent hydrocarbon radical, for example a C₁-C₂₀ alkyl radical.

In some embodiments, the at least one aminated silicone of formula (IV) is chosen from compounds of formula (V):

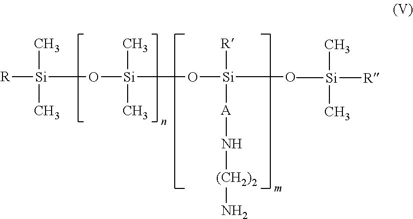

in which R, R′, R″, which may be identical or different, denote a C₁-C₄ alkyl radical, e.g., CH₃; a C₁-C₄ alkoxy radical, e.g., methoxy; or OH; A represents a linear or branched, C₃-C₈ alkylene radical, e.g., a C₃-C₆ alkylene radical; m and n are integers depending on the molecular weight and whose sum ranges from 1 and 2000.

According to a first possibility, R, R′, R″, which may be identical or different, represent a C₁-C₄ alkyl radical or a hydroxyl radical, A represents a C₃ alkylene radical and

m and n are such that the weight-average molecular weight of the compound ranges from about 5000 and 500,000. The compounds of this type are called “amodimethicone” in the CTFA dictionary.

According to a second possibility, R, R′, R″, which may be identical or different, represent a C₁-C₄ alkoxy radical or a hydroxyl radical, at least one of the radicals R or R″ is an alkoxy radical and A represents a C₃ alkylene radical. The hydroxy/alkoxy molar ratio, in some embodiments, ranges from 0.2/1 to 0.4/1, for example, equal to 0.3/1. Moreover, m and n are such that the weight-average molecular weight of the compound ranges from 2,000 to 10⁶. In some embodiments, n ranges from 0 to 999 and m ranges from 1 to 1000, the sum of n and m ranging from 1 to 1000.

This category of compounds includes, e.g., the product Belsil®ADM 652, marketed by Wacker.

According to a third possibility, R, R″, which are different, represent a C₁-C₄ alkoxy radical or a hydroxyl radical, at least one of the radicals R, R″ is an alkoxy radical, R′ represents a methyl radical and A represents a C₃ alkylene radical. The hydroxy/alkoxy molar ratio may in some embodiments range from 1/0.8 to 1/1.1; for example, it may equal 1/0.95. Moreover, m and n are such that the weight-average molecular weight of the compound ranges from 2000 to 200,000. In some embodiments, n ranges from 0 and 999 and m ranges from 1 and 1000, the sum of n and m ranging from 1 to 1000.

An example is the product FLUIDWR® 1300, marketed by Wacker.

Note that the molecular weight of these silicones is determined by gel permeation chromatography (room temperature, polystyrene standard; μ styragem columns; eluent THF; flow of 1 mm/m; inject 200 μl of a solution at 0.5 wt. % of silicone in THF and use refractometry and a UV-meter for detection).

An example of a product of formula (IV) is the polymer designated “trimethylsilylamodimethicone” in the CTFA dictionary, of formula (VI):

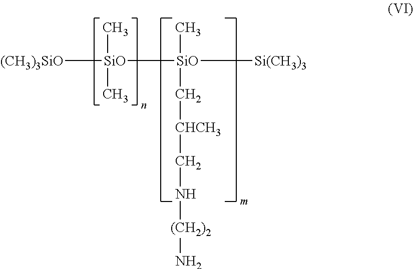

in which m and n are numbers that sum to a value ranging from 1 to 2000, e.g., from 50 to 150; in some embodiments, n can denote a number from 0 to 1999, or from 49 to 149, and/or m can denote a number from 1 to 2000, or from 1 to 10.

Such compounds are described for example in EP 95238; a compound of formula (IV) is for example sold under the name Q2-8220 by the company OSI.

b) aminated silicones of formula (VII):

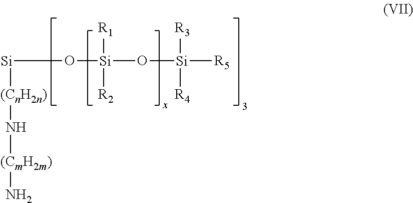

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5,

and in which x is chosen in such a way that the amine index ranges from 0.01 to 1 meq/g.

Other aminated silicones can be used within the scope of the disclosure such as the product referred to in the CTFA dictionary by the name POLYSILICONE-9 (CAS No. 165445-18-1).

In some embodiments, the at least one aminated silicone used in the cosmetic compositions according to the disclosure is chosen from silicones of formula (VI).

The at least one aminated silicone used in the compositions according to the disclosure can be present in an amount ranging from 0.1 to 10 wt. %, such as in an amount ranging from 0.2 to 5 wt. %, or in an amount ranging from 0.3 to 3 wt. %, relative to the total weight of the composition.

The cosmetic compositions according to the disclosure can also comprise at least one organic acid.

“Organic acid” means any non-polymeric organic compound having at least one acid functional group chosen from carboxylic acid, sulphonic acid, phosphoric acid functional groups.

In some embodiments, the at least one organic acid is not a surfactant.

In some embodiments, the molecular weight of the at least one organic acid is less than 250 atomic mass units, or less than 200 atomic mass units.

The at least one organic acid can comprise at least one amino acid.

In some embodiments, the at least one organic acid is chosen from acetic acid, propanoic acid, butanoic acid, lactic acid, malic acid, glycolic acid, ascorbic acid, maleic acid, phthalic acid, succinic acid, taurine, tartaric acid, arginine, glycine, glucuronic acid, gluconic acid and citric acid.

In some embodiments, the at least one organic acid according to the disclosure is chosen from carboxylic acids, such as alpha-hydroxylated or AHA carboxylic acids.

In some embodiments, the at least one organic acid used in compositions according to the disclosure is chosen from lactic acid and citric acid.

In the compositions, the at least one organic acid can be in free form or salified.

The at least one organic acid that can be used in the compositions according to the present disclosure can be present at a content expressed as free acids ranging from 0.01 to 10 wt. %, such as at a content ranging from 0.1 to 8 wt. %, or from 0.2 to 5 wt. %, relative to the total weight of the composition.

The compositions according to the present disclosure can also comprise at least one additional surfactant chosen from amphoteric and non-ionic surfactants.

The amphoteric or zwitterionic surfactants usable in the present disclosure can be, for example, derivatives of secondary or tertiary aliphatic amines, in which the aliphatic group is a linear or branched chain having from 8 to 22 carbon atoms and containing at least one anionic group such as, for example, a carboxylate, sulphonate, sulphate, phosphate or phosphonate group. Examples include alkyl(C₈₋₂₀)betaines, sulphobetaines, (C₈₋₂₀ alkyl)amido(C₆₋₈ alkyl)betaines or (C₈₋₂₀ alkyl)amido(C₆₋₈ alkyl)sulphobetaines.

Among the amine derivatives, examples include the products marketed under the name MIRANOL®, as described in U.S. Pat. No. 2,528,378 and U.S. Pat. No. 2,781,354 and classified in the CTFA dictionary, 3rd edition, 1982, under the designations Amphocarboxyglycinate and Amphocarboxypropionate with the respective structures (VIII) and (IX):

Rₐ—CONHCH₂CH₂—N(R_{b})(R_{c})(CH₂COO′) (VIII)

in which:

Rₐ represents an alkyl group derived from an Rₐ—COOH acid present in hydrolyzed copra oil, a heptyl, nonyl or undecyl group,

R_{b} represents a beta-hydroxyethyl group, and

R_{c} represents a carboxymethyl group;

and

Rₐ′—CONHCH₂CH₂—N(B)(B′) (IX)

in which:

B represents —CH₂CH₂OX′,

B′ represents —(CH₂)_{z}—Y′, with z=1 or 2,

X′ represents the group —CH₂CH₂—COOH or a hydrogen atom,

Y′ represents —COOH or the group —CH₂—CHOH—SO₃H,

Rₐ′ represents an alkyl group of an Rₐ′—COOH acid present in copra oil or in hydrolyzed linseed oil, an alkyl group, e.g., of C₁₇ and its iso form, an unsaturated C₁₇ group.

These compounds are classified in the CTFA dictionary, 5th edition, 1993, under the designations disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

A non-limiting example is the cocoamphodiacetate marketed by the company RHODIA under the trade name MIRANOL® C2M concentrate.

In some embodiments, the at least one amphoteric or zwitterionic surfactant is chosen from (C₈₋₂₀ alkyl)-betaines, (C₆₋₂₀ alkyl)-amido(C₆₋₈ alkyl)betaines and mixtures thereof.

When they are present, the amount of the at least one amphoteric or zwitterionic surfactant can in some embodiments range from 0.1 to 15 wt. %, from 0.5 to 10 wt. %, or from 1 to 8 wt. %, relative to the total weight of the composition.

Examples of additional non-ionic surfactants usable in the compositions of the present disclosure are described for example in “Handbook of Surfactants” by M. R. PORTER, Publ. Blackie & Son (Glasgow and London), 1991, pp 116-178. They can be chosen from, for example, alcohols, alpha-diols, alkyl(C₁₋₂₀)phenols or polyethoxylated, polypropoxylated or polyglycerolated fatty acids, with a fatty chain having, for example, from 8 to 18 carbon atoms, and the number of ethylene oxide or propylene oxide groups can in some embodiments range from 2 to 50 and the number of glycerol groups can in some embodiments range from 2 to 30.

Additional examples include the condensates of ethylene oxide and of propylene oxide on fatty alcohols; the polyethoxylated fatty amides in some embodiments have from 2 to 30 ethylene oxide units, the polyglycerolated fatty amides having on average from 1 to 5 glycerol groups or from 1.5 to 4 glycerol groups, the ethoxylated esters of sorbitan fatty acids having from 2 to 30 ethylene oxide units, the esters of sucrose fatty acids, the esters of fatty acids of polyethylene glycol, the (C₆₋₂₄ alkyl)polyglycosides, the derivatives of N—(C₆₋₂₄ alkyl)glucamine, the oxides of amines such as the oxides of (C₁₀₋₁₄ alkyl)amines or oxides of N—(C₁₀₋₁₄ acyl)-aminopropylmorpholine.

When they are present, the amount of the additional non-ionic surfactant(s) can in some embodiments range from 0.01 to 20 wt. %, or from 0.1 to 10 wt. % relative to the total weight of the composition.

In some embodiments, the total amount of surfactants in the cosmetic compositions according to the disclosure ranges from 3 to 50 wt. %, from 5 to 30 wt. %, or from 8 to 20 wt. %, relative to the total weight of the cosmetic composition.

The cosmetic compositions can also comprise at least one cationic polymer.

In the sense of the present disclosure, the expression “cationic polymer” denotes any polymer containing cationic groups and/or groups that are ionizable to cationic groups.

The cationic polymers optionally present in the compositions according to the disclosure can be chosen from all those already known per se as improving the cosmetic properties of the hair, for example those described in patent application EP-A-337 354 and in French patents FR-2 270 846, 2 383 660, 2 598 611, 2 470 596 and 2 519 863.

The cationic polymers can be chosen from those that contain units having primary, secondary, tertiary and/or quaternary amine groups, which can either form part of the main polymer chain, or can be carried by a side substituent directly joined to the latter.

The cationic polymers used can have a number-average molecular weight ranging from about 500 to 5×10⁶ atomic mass units, or from 10³ to 3×10⁶ atomic mass units.

Examples of cationic polymers include polymers of the polyamine, polyaminoamide and quaternary polyammonium type.

These products are described, for example, in French patents No. 2 505 348 or 2 542 997. Among said polymers, non-limiting examples include:

(1) The homopolymers or copolymers derived from acrylic or methacrylic esters or amides and having at least one of the units of formulae (X), (XI), (XII) or (XIII):

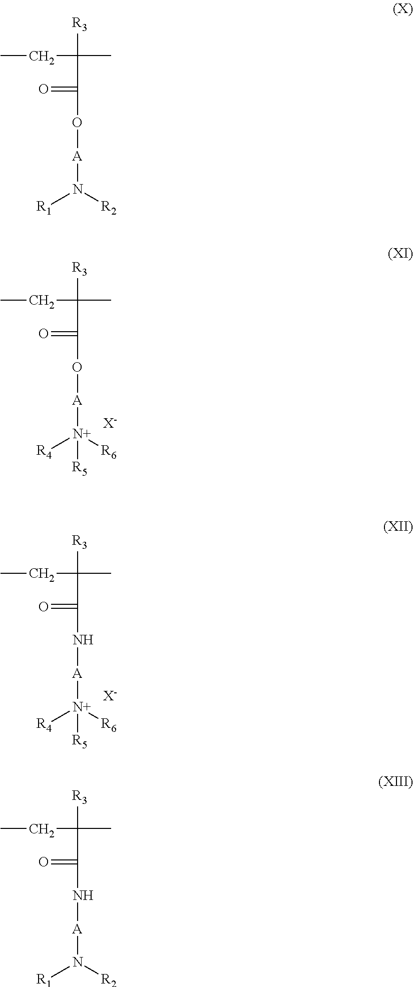

in which:

R₃, which may be identical or different, denote a hydrogen atom or a CH₃ radical;

A, which may be identical or different, represent a linear or branched alkyl group, of 1 to 6 carbon atoms, such as 2 or 3 carbon atoms, or a hydroxyalkyl group with 1 to 4 carbon atoms;

R₄, R₅, R₆, which may be identical or different, represent an alkyl group having from 1 to 18 carbon atoms or a benzyl radical, such as an alkyl group having from 1 to 6 carbon atoms;

R₁ and R₂, which may be identical or different, represent hydrogen or an alkyl group having from 1 to 6 carbon atoms such as methyl or ethyl;

X⁻ denotes an anion derived from a mineral or organic acid such as a methosulphate anion or a halide such as chloride or bromide.

An example is the homopolymer ethyl trimethyl ammonium methacrylate chloride.

The polymers of family (1) can further contain at least one unit derived from comonomers, which can be chosen from the group comprising acrylamides, methacrylamides, acrylamide diacetones, acrylamides and methacrylamides substituted on the nitrogen with lower (C₁-C₄) alkyls, acrylic or methacrylic acids or their esters, vinyllactams such as vinylpyrrolidone or vinylcaprolactam, and vinyl esters.

Thus, among these polymers of family (1), examples include:

copolymers of acrylamide and of dimethylaminoethyl methacrylate quaternized at the dimethyl sulphate or with a dimethyl halide, such as that sold under the name HERCOFLOC by the company HERCULES,

copolymers of acrylamide and of methacryloyloxyethyltrimethylammonium chloride described for example in patent application EP-A-080976 and sold under the name BINA QUAT P 100 by the company CIBA GEIGY,

the copolymer of acrylamide and of methacryloyloxyethyltrimethylammonium methosulphate sold under the name RETEN by the company HERCULES,

the vinylpyrrolidone/acrylate copolymers or dialkylaminoalkyl methacrylate, quaternized or not, such as the products sold under the name “GAFQUAT” by the company ISP for example “GAFQUAT 734” or “GAFQUAT 755” or else the products designated “COPOLYMER 845, 958 and 937”. These polymers are described in detail in French patents 2,077,143 and 2,393,573,

the terpolymers dimethyl amino ethyl methacrylate/vinylcaprolactam/vinylpyrrolidone such as the product sold under the name GAFFIX VC 713 by the company ISP,

the vinylpyrrolidone/methacrylamidopropyl dimethylamine copolymer marketed under the name STYLEZE CC 10 by ISP,

the quaternized vinylpyrrolidone/dimethylaminopropyl methacrylamide copolymers such as the product sold under the name “GAFQUAT HS 100” by the company ISP,

the crosslinked polymers of methacryloyloxyalkyl(C₁-C₄) trialkyl(C₁-C₄)ammonium salts such as the polymers obtained by homopolymerization of dimethylaminoethylmethacrylate quaternized by methyl chloride, or by copolymerization of acrylamide with dimethylaminoethylmethacrylate quaternized by methyl chloride, the homo- or copolymerization being followed by crosslinking by a compound with an olefinic unsaturation, such as methylene bis acrylamide. In some embodiments, a crosslinked acrylamide/methacryloyloxyethyl trimethylammonium chloride copolymer (20/80 in weight) can be used in the form of a dispersion containing 50 wt. % of said copolymer in mineral oil. This dispersion is marketed under the name “SALCARE® SC 92” by the company CIBA. It is also possible to use a crosslinked homopolymer of methacryloyloxyethyl trimethylammonium chloride containing about 50 wt. % of the homopolymer in mineral oil or in a liquid ester. These dispersions are marketed under the names “SALCARE® SC 95” and “SALCARE® SC 96” by the company CIBA.

(2) The derivatives of cellulose ethers having quaternary ammonium groups described in French patent 1 492 597, such as the polymers marketed under the names “UCARE POLYMER JR” (JR 400, JR 125, JR 30M) or “LR” (LR 400, LR 30M) by the company AMERCHOL. These polymers are also defined in the CTFA dictionary as quaternary ammoniums of hydroxyethylcellulose that have reacted with an epoxide substituted with a trimethylammonium group.

(3) The cellulose copolymers or cellulose derivatives grafted with a water-soluble monomer of quaternary ammonium, for example, those described in U.S. Pat. No. 4,131,576, such as the hydroxyalkyl celluloses, for instance the hydroxymethyl, hydroxyethyl or hydroxypropyl celluloses grafted with a methacryloylethyl trimethylammonium salt, a methacrylamidopropyl trimethylammonium salt, or dimethyl-diallylammonium salt.

The commercial products corresponding to this definition include, for example, the products sold under the name “CELQUAT L 200” and “CELQUAT H 100” by the company National Starch.

(4) The cationic guar gums described in U.S. Pat. Nos. 3,589,578 and 4,031,307 such as the guar gums containing trialkylammonium cationic groups. For example, guar gums modified by a 2,3-epoxypropyl trimethylammonium salt (e.g. chloride) can be used.

Examples of such products are marketed under the trade names JAGUAR C13S, JAGUAR C 15, JAGUAR C 17 or JAGUAR C162 by the company RHODIA.

(5) The polymers constituted of piperazinyl units and linear or branched alkylene or hydroxyalkylene divalent radicals, optionally interrupted by oxygen, sulphur, or nitrogen atoms or by aromatic or heterocyclic rings, as well as the products of oxidation and/or of quaternization of these polymers. Examples of such polymers are described, for example, in French patents 2,162,025 and 2,280,361.

(6) The water-soluble polyaminoamides, which can be prepared by polycondensation of an acid compound with a polyamine; these polyaminoamides can be crosslinked by an epihalohydrin, a diepoxide, a dianhydride, an unsaturated dianhydride, a bis-unsaturated derivative, a bis-halohydrin, a bis-azetidinium, a bis-haloacyldiamine, an alkyl bis-halide or by an oligomer resulting from the reaction of a reactive bifunctional compound on a bis-halohydrin, a bis-azetidinium, a bis-haloacyldiamine, an alkyl bis-halide, an epilhalohydrin, a diepoxide or a bis-unsaturated derivative; the crosslinking agent being used in proportions ranging from 0.025 to 0.35 mole per amine group of polyaminoamide; these polyaminoamides can be alkylated or, if they have at least one tertiary amine functional group, quaternized. Such polymers are described, for example, in French patents 2,252,840 and 2,368,508.

(7) The derivatives of polyaminoamides resulting from the condensation of polyamine polyalkylenes with polycarboxylic acids followed by alkylation by bifunctional agents. Examples include the adipic acid-diacoylaminohydroxyalkyldialoylene triamine polymers in which the alkyl radical has from 1 to 4 carbon atoms, such as methyl, ethyl, or propyl. Such polymers are described, for example, in French patent 1,583,363.

Among these derivatives, additional examples include the adipic acid/dimethylaminohydroxypropyl/diethylene triamine polymers sold under the name “CARTARETINE F, F4 or F8” by the company Sandoz.

(8) The polymers obtained by reaction of a polyalkylene polyamine having two primary amine groups and at least one secondary amine group with a dicarboxylic acid chosen from diglycolic acid and saturated aliphatic dicarboxylic acids having from 3 to 8 carbon atoms. The molar ratio of polyalkylene polylamine to dicarboxylic acid ranging from 0.8:1 to 1.4:1; the resultant polyaminoamide being reacted with epichlorohydrin in a molar ratio of epichlorohydrin to the secondary amine group of the polyaminoamide between 0.5:1 and 1.8:1. Such polymers are described, for example, in U.S. Pat. Nos. 3,227,615 and 2,961,347.

Examples of polymers of this type are marketed under the name “HERCOSETT 57” by the company Hercules Inc. and under the name “PD 170” or “DELSETTE 101” by the company Hercules in the case of the adipic acid/epoxypropyl/diethylene-triamine copolymer.

(9) The cyclopolymers of alkyl diallyl amine or of dialkyl diallyl ammonium such as the homopolymers or copolymers having, as the main constituent of the chain, units chosen from those of formulae (XIV) and (XV):

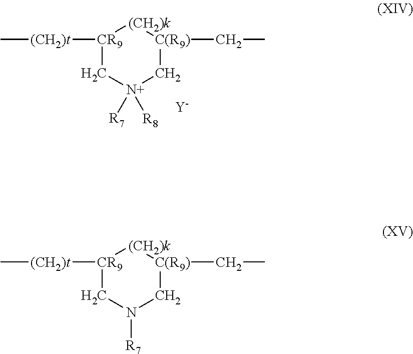

In these formulae, k and t are equal to 0 or 1, the sum k+t being equal to 1; R₉ denotes a hydrogen atom or a methyl radical; R₇ and R₈, independently of one another, denote an alkyl group having from 1 to 6 carbon atoms, or a group chosen from a hydroxyalkyl group in which the alkyl group has 1 to 5 carbon atoms or a lower (C₁-C₄) amidoalkyl group, or R₇ and R₈ can denote, together with the nitrogen atom to which they are attached, a heterocyclic group, such as piperidinyl or morpholinyl; in some embodiments, R₇ and R₈, independently of one another, denote an alkyl group having from 1 to 4 carbon atoms; Y⁻ is an anion such as bromide, chloride, acetate, borate, citrate, tartrate, bisulphate, bisulphite, sulphate, or phosphate. These polymers are described, for example, in French patent 2,080,759 and in its certificate of addition 2,190,406.

Additional examples include the homopolymer of dimethyldiallylammonium chloride sold under the name “MERQUAT 100” by the company NALCO (and its homologues of low weight-average molecular weight) and the copolymers of diallyldimethylammonium chloride and acrylamide marketed under the name “MERQUAT 550”, “MERQUAT 7SPR”.

(10) The polymer of quaternary diammonium containing repeat units of formula (XVI):

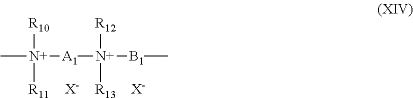

In formula (XVI):

R₁₀, R₁₁, R₁₂ and R₁₃, which may be identical or different, represent aliphatic, alicyclic, or arylaliphatic radicals containing from 1 to 6 carbon atoms or lower hydroxyalkylaliphatic radicals, or else R₁₀, R₁₁, R₁₂ and R₁₃, together or separately, constitute, with the nitrogen atoms to which they are attached, heterocycles optionally containing a second heteroatom other than nitrogen or else R₁₀, R₁₁, R₁₂ and R₁₃ represent a linear or branched C₁-C₆ alkyl radical substituted with a nitrile, ester, acyl, amide or —CO—O—R₁₄-D or —CO—NH—R₁₄-D group, where R₁₄ is an alkylene and D is a quaternary ammonium group;

A₁ and B₁ represent polymethylene groups containing from 2 to 8 carbon atoms which can be linear or branched, saturated or unsaturated, and which can contain, be bound to or intercalated in the main chain, at least one aromatic ring, or at least one oxygen or sulphur atom or sulphoxide, sulphone, disulphide, amino, alkylamino, hydroxyl, quaternary ammonium, ureido, amide or ester group, and

X⁻ denotes an anion derived from a mineral or organic acid;

A₁, R₁₀ and R₁₂ can form, with the two nitrogen atoms to which they are attached, a piperazine ring; if A₁ denotes a linear or branched, saturated or unsaturated alkylene or hydroxyalkylene radical, B1 can also denote a group —(CH₂)n-CO-D-OC—(CH₂)n- in which D denotes:

a) a glycol residue of formula: —O—Z—O—, where Z denotes a linear or branched hydrocarbon radical or a group of one of the following formulae:

—(CH₂—CH₂—O)x-CH₂—CH₂—

—[CH₂—CH(CH₃)—O]y-CH₂—CH(CH₃)—

where x and y denote an integer from 1 to 4, representing a specified unique degree of polymerization or any number from 1 to 4 representing an average degree of polymerization;

b) a bis-secondary diamine residue such as a derivative of piperazine;

c) a bis-primary diamine residue of formula: —NH—Y—NH—, where Y denotes a linear or branched hydrocarbon radical, or else the divalent radical

—CH₂—CH₂—S—S—CH₂—CH₂—;

d) a ureylene group of formula: —NH—CO—NH—.

In some embodiments, X⁻ is an anion such as chloride or bromide.

In some embodiments, these polymers have a number-average molecular weight ranging from 1,000 to 100,000.

Polymers of this type are described, for example, in French patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is also possible to use the polymers that are constituted of repeat units of formula (XVI′):

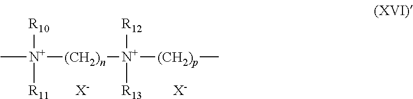

in which R₁₀, R₁₁, R₁₂ and R₁₃, which may be identical or different, denote an alkyl or hydroxyalkyl radical having from about 1 to 4 carbon atoms, n and p are integers ranging from 2 to 8 and, X⁻ is an anion derived from a mineral or organic acid. An example is MEXOMER PO marketed by the company CHIMEX.

(11) The quaternary polyammoniums constituted of repeat units of formula (XVII):

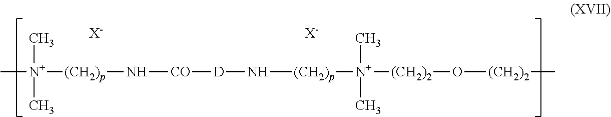

in which p denotes an integer ranging from about 1 to 6, D can be a bond between the neighboring moieties or can represent a group —(CH₂)ᵣ—CO— in which r denotes a number equal to 4 or to 7, X⁻ is an anion.

Such polymers can be prepared according to the methods described in U.S. Pat. Nos. 4,157,388, 4,702,906, and 4,719,282. They are also described in patent application EP-A-122 324.

Examples include the products “MIRAPOL A 15”, “MIRAPOL AD1”, “MIRAPOL AZ1” and “MIRAPOL 175” sold by the company Miranol.

(12) The quaternary polymers of vinylpyrrolidone and of vinylimidazole such as the products marketed under the names LUVIQUAT FC 905, FC 550 and FC 370 by the company BASF. These polymers can also comprise other monomers such as diallyldialkylammonium halides. An example is the product marketed under the name LUVIQUAT SENSATION by the company BASF.

(13) The polyamines such as POLYQUART H sold by HENKEL, referred to as “Polyethylene Glycol (15) tallow polyamine” in the CTFA dictionary, or the polyamines of ethoxylated copra (15 OE).

Other cationic polymers usable within the scope of the disclosure are polyalkyleneimines, such as polyethyleneimines, polymers containing vinylpyridine or vinylpyridinium units, condensates of polyamines and of epichlorohydrin, quaternary polyureylenes and derivatives of chitin.

In some embodiments, the compositions according to the disclosure comprise at least one cationic polymer chosen from the polymers of families (1), (2), (3), (4), (9), (10) and (12).

In some embodiments, the at least one cationic polymer is chosen from cationic celluloses and cationic guar gums.

In some embodiments, the at least one cationic polymer is chosen from cationic celluloses, cationic guar gums and quaternary polymers of vinylpyrrolidone and of vinyl imidazole optionally combined with other monomers.

In some embodiments, the at least one cationic polymer is chosen from hydroxyalkylcelluloses, such as hydroxymethyl, hydroxyethyl or hydroxypropyl celluloses grafted with a methacryloylethyl trimethylammonium salt, a methacrylamidopropyltrimethylammonium salt, a dimethyldiallyl ammonium salt, cationic guar gums, the copolymer of vinylpyrrolidone and of vinylimidazole and dimethyldiallylammonium chloride.

The content of the at least one cationic polymer in the compositions according to the disclosure can range from 0.01 to 5 wt. % relative to the total weight of the composition, from 0.1 to 1 wt. %, or from 0.15 to 0.5 wt. %.

“Cosmetically acceptable medium” means a medium compatible with keratin fibers, such as the hair.

The cosmetically acceptable medium is constituted of water or a mixture of water and at least one cosmetically acceptable solvent chosen from C₁-C₄ lower alcohols, such as ethanol, isopropanol, tert-butanol or n-butanol; polyols such as glycerol, propylene glycol and polyethylene glycols; and mixtures thereof.

In some embodiments, the pH of the compositions according to the disclosure ranges from 3 to 11, from 5 to 10, or from 7 to 10.

The compositions according to the disclosure can further comprise at least one conventional additive known in the art, such as: thickeners or viscosity regulators, natural or synthetic; C₁₂-C₃₀ fatty alcohols; ceramides; oily fatty esters such as isopropyl myristate, myristyl myristate, cetyl palmitate and stearyl stearate; mineral, vegetable or synthetic oils such as α-olefins or palm oil; vitamins or provitamins; amphoteric polymers; pH stabilizers, preservatives, silicones other than the aminated silicones of the disclosure and colorants.

The thickener or thickeners can be chosen from cellulosic thickeners, for example hydroxyethylcellulose, hydroxypropylcellulose and carboxymethylcellulose, guar gum and its derivatives, for example hydroxypropyl guar, marketed by the company RHODIA under reference JAGUAR HP 105, gums of microbial origin, such as xanthan gum and scleroglucan gum, synthetic thickeners such as crosslinked homopolymers of acrylic acid or acrylamidopropanesulphonic acid, for example Carbomer, non-ionic, anionic, cationic or amphoteric associative polymers, such as the polymers marketed under the names PEMULEN TR1 or TR2 by the company GOODRICH, SALCARE SC90 by the company CIBA, ACULYN 22, 28, 33, 44 or 46 by the company ROHM & HAAS and ELFACOS T210 and T212 by the company AKZO.

A person skilled in the art will take care to select any additive(s) and their amount so that they do not adversely affect the properties of the compositions of the present disclosure.

These additives can be present in the compositions according to the disclosure in an amount, for example, ranging from 0 to 20 wt. % relative to the total weight of the composition.

In some embodiments, the cosmetic compositions of the disclosure are transparent or translucent, i.e. these compositions have a transmittance at 600 nanometres above 85%, above 90% or above 94%.

The compositions according to the disclosure can be used as shampoos for washing and conditioning the hair, for example, they can be applied on hair that is wet in effective amounts for washing it, and the foam generated by massaging or rubbing with the hands can then be removed, optionally after a pause, by rinsing with water, an operation that can be repeated one or more times.

Thus the present disclosure also relates to the use of a cosmetic composition as described previously, as shampoo, for cleaning and conditioning keratin fibers, e.g., human keratin fibers such as the hair.

Another aspect of the disclosure is a method of cosmetic treatment of keratin fibers, such as the hair, which comprises or consists of applying an effective amount of a composition as described above, on said fibers, and optionally rinsing after an optional leave-on period or pause.

In some embodiments, the method of cosmetic treatment of keratin fibers is a method for washing and conditioning keratin fibers, such as the hair.

The following examples serve to illustrate the present disclosure.

### EXAMPLES

### Example 1

Composition (A) according to the disclosure was prepared from the ingredients shown in the following table, the amounts being expressed as percentage by weight of product, relative to the total weight of the composition.

| | A |
|---|---|
| Composition | (disclosure) |
| Lactic acid | 0.27 |
| Mixture of chloro-5-methyl-2-isothiazoline-4-one-3/ | 0.1 |
| methylisothiazoline-4-one-3/magnesium chloride | |
| and nitrate⁽¹⁾ | |
| Oleate of polyethoxylated propylene glycol (55 OE) | 0.6 |
| and of propylene glycol in hydroglycolic solution⁽²⁾ | |
| Hydroxyethyl cellulose quaternized by 2,3- | 0.6 |
| epoxypropyl trimethyl ammonium chloride at 95.5% | |
| AS⁽³⁾ | |
| Polydimethyl siloxane with aminoethyl aminoisobutyl | 1 |
| and trimethylsiloxy groups⁽⁴⁾ | |
| 3-Aminopropyltriethoxysilane⁽⁵⁾ | 0.75 |
| Cocoyl betaine in aqueous solution at 30% AS⁽⁶⁾ | 17 |
| Cetyl alcohol, ethoxylated (20 OE) and propoxylated | 0.5 |
| (5 OP)⁽⁷⁾ | |
| Lauryl ether carboxylic acid (4.5 OE) at 90% AS⁽⁸⁾ | 1 |
| Monoisopropanolamide of copra acids⁽⁹⁾ | 0.85 |
| Sodium lauryl ether sulphate (2.2 OE) in aqueous | 16 |
| solution (70% AS)⁽¹⁰⁾ | |
| pH adjuster | q.s. pH = 9 |
| Perfume | 0.5 |
| Deionized water | q.s. 100 g |

⁽¹⁾sold under the trade name KATHON CG by the company ROHM and HAAS
⁽²⁾sold under the trade name ANTIL 141 LIQUID by the company EVONIK GOLDSCHMIDT,
⁽³⁾sold under the trade name POLYQUAT 400 KC by the company KCI,
⁽⁴⁾sold under the trade name DC2 8566 AMINOFLUID by the company DOW CORNING,
⁽⁵⁾sold under the name XIAMETER OFS 6011 SILANE by the company DOW CORNING,
⁽⁶⁾sold under the name MIRATAINE BB/FLA by the company RHODIA,
⁽⁷⁾sold under the name PROCETYL AWS-LQ by the company CRODA,
⁽⁸⁾sold under the name AKYPO RLM 45 CA by the company KAO,
⁽⁹⁾sold under the name EMPILAN CIS by the company HUNTSMAN,
⁽¹⁰⁾sold under the name TEXAPON AOS 225UP by the company COGNIS

A composition was obtained that was clear and stable over time.

Applied as shampoo, composition (A) endowed the hair with weight, volume and a satisfactory soft feel.

### Example 2

Composition (B) according to the disclosure was prepared from the ingredients shown in the following table, the amounts being expressed as percentage by weight of active substances, relative to the total weight of the composition.

| Composition | B (disclosure) |
|---|---|
| Sodium chloride | 1.436 |
| Lactic acid | 0.2 |
| Ethylene glycol distearate | 1.6 |
| Hydroxypropylguar trimethyl ammonium chloride⁽¹⁾ | 0.2 |
| Carboxyvinyl polymer⁽²⁾ | 0.15 |
| Polydimethylsiloxane with aminoethyl iminopropyl | 1.7 |
| groups with methoxy and/or hydroxy functional | |
| group and alpha-omega silanols in cationic aqueous | |
| emulsion at 60%⁽³⁾ | |
| Hexylene glycol | 0.5 |
| 3-Aminopropyltriethoxysilane⁽⁷⁾ | 0.75 |
| Cocoyl betaine in aqueous solution at 30% AS⁽⁵⁾ | 6 |
| Monoisopropanolamide of copra acids⁽⁶⁾ | 0.7 |
| Sodium lauryl ether sulphate (2.2 OE) in aqueous | 17.4 |
| solution (70% AS)⁽⁷⁾ | |
| pH adjuster | q.s. pH = 9 |
| Perfume | 0.5 |
| Deionized water | q.s. 100 g |

⁽¹⁾sold under the trade name JAGUAR C13S by the company RHODIA
⁽²⁾sold under the trade name CARBOPOL 980 POLYMER by the company LUBRIZOL,
⁽³⁾sold under the trade name DC2-8299 CATIONIC EMULSION by the company DOW CORNING,
⁽⁴⁾sold under the trade name XIAMETER OFS 6011 SILANE by the company DOW CORNING,
⁽⁵⁾sold under the name MIRATAINE BB/FLA by the company RHODIA,
⁽⁶⁾sold under the name EMPILAN CIS by the company HUNTSMAN
⁽⁷⁾sold under the name TEXAPON AOS 225UP by the company COGNIS

A nacreous composition was obtained, which was stable over time.

Applied as shampoo, composition (B) endowed the hair with weight, volume, and a satisfactory soft feel.

What is claimed is:

1. A cosmetic composition for cleaning and conditioning keratin fibers comprising, in a cosmetically acceptable medium,
i) at least one organic silicon compound chosen from silanes comprising one silicon atom and siloxanes comprising two or three silicon atoms, said at least one organic silicon compound having at least one basic chemical functional group and at least one group chosen from hydroxyl groups and hydrolysable groups per molecule;
ii) at least one anionic surfactant; and
iii) at least one non-quaternized aminated silicone different from the at least one organic silicon compound of part i).

2. The cosmetic composition of claim 1, wherein the at least one basic chemical functional group of the at least one organic silicon compound is chosen from primary, secondary and tertiary amines.

3. The cosmetic composition of claim 1, wherein the at least one organic silicon compound comprises at least one hydrolysable group chosen from alkoxy, aryloxy and halogen groups.

4. The cosmetic composition according to claim 1, wherein the at least one organic silicon compound is chosen from:
compounds of formula (I): 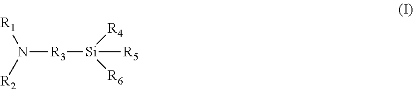
in which:
R₄ represents a halogen, OR′ or R′₁;
R₅ represents a halogen, OR″ or R′₂;
R₆ represents a halogen, OR′″ or R′₃;
R₃, R′₁, R′₂, R′₃ represent, independently of one another, a saturated or unsaturated, linear or branched hydrocarbon group, optionally bearing additional chemical groups;
R₁, R₂, R′, R″ and R′″ represent, independently of one another, hydrogen or a saturated or unsaturated, linear or branched hydrocarbon group, optionally bearing additional chemical groups;
at least two of the groups R₄, R₅ and R₆ denote respectively OR′, OR″ and OR′″, at least two of the groups R′, R″ and R′″ being different from hydrogen;
and compounds of formula (II): 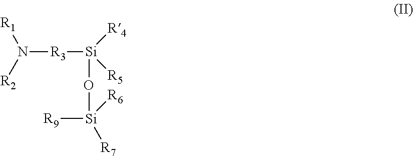
in which:
R₁, R₂, R₃, R₅ and R₆ are defined as for the compounds of formula (I);
R′₄ represents a halogen, or OR₁₁;
R₇ represents a halogen, OR₁₀ or R″₁;
R₉ represents a halogen, OR₈, R″₂ or R₃NR₁R₂;
R″₁ and R″₂ represent, independently of one another, a saturated or unsaturated, linear or branched hydrocarbon group, optionally bearing additional chemical groups; and
R₁₁, R₁₀ and R₈ represent, independently of one another, a hydrogen atom or a saturated or unsaturated, linear or branched hydrocarbon group, optionally bearing additional chemical groups,
and at least one of groups R₆, R₇ and R₉ denote a halogen atom, OR′″, OR₁₀ or OR₈.

5. The cosmetic composition of claim 4, wherein groups R₁, R₂, R′, R′₁, R′₂, R′₃, R″, R′″, R″₁, R″₂, R₈, R₁₀ and R₁₁ are chosen from C₁-C₁₂ alkyl, C₆ to C₁₄ aryl, C₁ to C₈ alkyl-C₆ to C₁₄ aryl, and C₆ to C₁₄ aryl-C₁ to C₈ alkyl radicals.

6. The cosmetic composition according to claim 1, wherein the at least one organic silicon compound is chosen from compounds of formula (III): 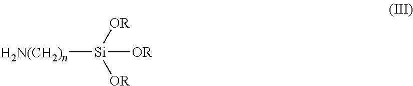
in which the radicals R, which may be identical or different, are chosen from C₁-C₆ alkyl radicals and n is an integer ranging from 1 to 6.

7. The cosmetic composition of claim 6, wherein n is an integer ranging from 2 to 4.

8. The cosmetic composition according to claim 1, wherein the at least one anionic surfactant is chosen from alkylsulphates, alkylethersulphates and alkylethercarboxylates.

9. The cosmetic composition of claim 8, wherein the at least one anionic surfactant chosen from alkylsulphates, alkylethersulphates and alkylethercarboxylates is in a form chosen from salts of alkali metals, of alkaline-earth metals, of ammonium, of amines, or of amino alcohols.

10. The cosmetic composition of claim 1, wherein the at least one aminated silicone is chosen from:
(a) compounds of formula (IV):
(R¹)ₐ(T)₃₋ₐ-Si[OSi(T)_{2]}ₙ-[OSi(T)_{b}(R¹)_{2-b}]ₘ—OSi(T)₃₋ₐ-(R¹)ₐ (IV)
in which:
T is a hydrogen or a phenyl, hydroxyl (—OH), or C₁-C₈ alkyl radical,
a denotes an integer from zero to 3,
b denotes zero or 1,
m and n are numbers that sum to a value ranging from 1 to 2,000;
R¹ is a monovalent radical of formula —C_{q}H_{2q}L in which q is a number ranging from 2 to 8 and L is an amino group chosen from the groups:
—N(R²)—CH₂—CH₂—N(R²)₂;
—N(R²)₂;
in which R² denotes a hydrogen atom, a phenyl radical, a benzyl radical, or a saturated monovalent hydrocarbon radical;
(b) aminated silicones of formula (VII): 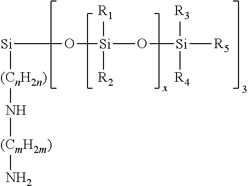
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5,
and in which x is chosen in such a way to give an amine index ranging from 0.01 to 1 meq/g; and
(c) polysilicone-9.

11. The cosmetic composition of claim 10, wherein the at least one aminated silicone is chosen from compounds of formula (IV), and at least one of the following is true:
T is methyl or C₁-C₈ alkoxy,
a denotes the number 0,
b denotes the number 1,
m and n are numbers that sum to a value ranging from 50 to 150, or
R² denotes a C₁-C₂₀ alkyl radical.

12. The cosmetic composition of claim 11, wherein at least one of the following is true:
T is methoxy;
n denotes a number ranging from 49 to 149; or
m denotes a number ranging from 1 to 10.

13. The cosmetic composition of claim 10, wherein the at least one aminated silicone is chosen from aminated silicones of formula (V): 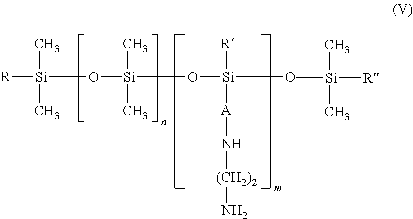
in which:
R, R′, R″, which may be identical or different, denote a C₁-C₄ alkyl radical, a C₁-C₄ alkoxy radical, or OH;
A represents a linear or branched C₃-C₈ alkylene radical; and
m and n are integers whose sum ranges from 1 to 2000.

14. The cosmetic composition of claim 13, wherein:
at least one of R, R′, R″, which may be identical or different, denotes CH₃ or methoxy; and/or
A represents a linear or branched C₃-C₆ alkylene radical.

15. The cosmetic composition of claim 10, wherein the at least one aminated silicone is chosen from aminated silicones of formula (IV) which are trimethylsilylamodimethicones of formula (VI): 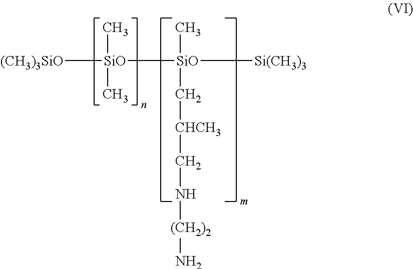
in which m and n are numbers that sum to a value ranging from 1 to 2,000.

16. The cosmetic composition of claim 1, further comprising at least one organic acid.

17. The cosmetic composition of claim 1, further comprising at least one additional surfactant chosen from amphoteric surfactants and non-ionic surfactants.

18. The cosmetic composition of claim 1, further comprising, in addition to the at least one organic silicon compound and the at least one non-quaternized aminated silicone, which may or may not be cationic, at least one cationic polymer.

19. The cosmetic composition of claim 1, wherein the composition is formulated as a shampoo.

20. The cosmetic composition of claim 1, wherein the composition is formulated for cleaning and conditioning human hair.

21. A method of cosmetic treatment of keratin fibers, comprising:
applying a cosmetic composition on said fibers, wherein:
the cosmetic composition comprises, in a cosmetically acceptable medium,
i) at least one organic silicon compound chosen from silanes comprising one silicon atom and siloxanes comprising two or three silicon atoms, said organic silicon compound having at least one basic chemical functional group and at least one group chosen from hydroxyl groups and hydrolysable groups per molecule;
ii) at least one anionic surfactant; and
iii) at least one non-quaternized aminated silicone different from the at least one organic silicon compound of part i); and
optionally rinsing one or more times after an optional leave-on period.

22. The method of claim 21, wherein the keratin fibers comprise human hair.
